(19) United States
(12) Patent Application Publication
(10) Pub. No.: US 2013/0195978 A1
(43) Pub. Date: Aug. 1, 2013

(54) Darunavir Compositions

(71) Applicants: Bandi Parthasarashi Reddy, Andhrapradesh (IN); Podili Khadgapathi, Andhrapradesh (IN); Goli Kamalakar Reddy, Andhrapradesh (IN)

(72) Inventors: Bandi Parthasarashi Reddy, Andhrapradesh (IN); Podili Khadgapathi, Andhrapradesh (IN); Goli Kamalakar Reddy, Andhrapradesh (IN)

(73) Assignee: HETERO RESEARCH FOUNDATION, Hyderabad (IN)

(21) Appl. No.: 13/696,702

(22) PCT Filed: May 10, 2010
(86) PCT No.: PCT/IN10/00299

§ 371 Date: Mar. 14, 2013

(51) Int. Cl. A61K 9/20

(52) U.S. Cl. CPC A61K 9/2072
USPC 424/474; 514/470; 424/400; 428/402

(57) ABSTRACT

The present invention relates to an oral pharmaceutical composition of amorphous darunavir.

### FIELD OF THE INVENTION

The present invention relates to an oral pharmaceutical composition of amorphous darunavir.

### BACKGROUND OF THE INVENTION

Darunavir, also known as TMC-114 and UIC-94017, is a HIV-1 protease inhibitor. It selectively inhibits the cleavage of HIV encoded Gag-Pol polyproteins in infected cells, thereby preventing the formation of mature virus particles.

Darunavir is chemically [(3R,3aS,6aR)-2,3,3a,4,5,6a-Hexahydrofuro[5,4-b]furan-3-yl]N-[(2S,3R)-4-[(4-aminophenyl)sulfonyl-(2-methylpropyl)amino]-3-hydroxy-1-phenylbutan-2-yl]carbamate. Its empirical formula is C₂₇H₃₇N₃O₇S, and its molecular weight is 547.66. Darunavir has the following structural formula.

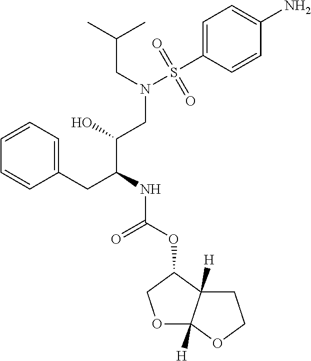 [SMILES: CC(C)CN(C[C@@H](O)[C@H](Cc1ccccc1)NC(=O)O[C@H]1CO[C@H]2OCC[C@H]21)S(=O)(=O)c1ccc(N)cc1]

Darunavir is commercially available as tablets containing darunavir ethanolate under the trade name PREZISTA® in the United States, Europe and Canada.

The synthesis of darunavir and the manner in which it may be used to treat HIV infection are described in U.S. Pat. Nos. 5,843,946, 6,248,775 and 6,335,460.

The present invention relates to an oral pharmaceutical composition comprising amorphous darunavir having d₉₀ particle size of about 150 μm to about 250 μm.

### OBJECTIVE OF THE INVENTION

Accordingly, the main objective of the invention is to provide an oral pharmaceutical composition comprising amorphous darunavir having a d₉₀ particle size of about 150 μm to about 250 μm.

### DETAILED DESCRIPTION OF THE INVENTION

According to the present invention there is provided an oral pharmaceutical composition comprising amorphous darunavir having a d₉₀ particle size of about 150 μm to 250 μm.

The preferable d₉₀ particle size of darunavir is in the range of 175 to 225 μm.

More preferably, the d₉₀ particle size of darunavir is in the range of 190 to 200 μm.

Preferably, the oral pharmaceutical composition is a solid oral dosage form.

More preferably, the solid dosage form is in the form of tablet.

The tablet composition is optionally film coated.

The oral pharmaceutical composition of amorphous darunavir may be prepared by direct compression, wet granulation or roll compaction.

Preferably oral pharmaceutical composition of amorphous darunavir may be prepared by direct compression.

The oral pharmaceutical composition of the present invention may contain one or more additional excipients. These excipients may be selected from diluents, binders, disintegrants and lubricants.

Preferably, the diluent is selected from lactose, sucrose, glucose, mannitol, sorbitol, calcium carbonate, microcrystalline cellulose, Prosolv, magnesium stearate and mixtures thereof. More preferably, the diluent is Prosolv.

The preferable binder is selected from L-Hydroxy propyl cellulose, polyvinyl pyrrolidine, hydroxyl propyl methyl cellulose, hydroxyl ethyl cellulose and pre-gelatinized starch.

Preferably, the disintegrant is selected from croscarmellose sodium, crospovidone, sodium starch glycolate and low substituted hydroxyl propyl cellulose.

The more preferable disintegrant is selected from low substituted hydroxyl propyl cellulose and crospovidone.

Preferably, the lubricant is selected from sodium stearyl fumarate, magnesium stearate, zinc stearate, calcium stearate, stearic acid, talc, Glyceryl behenate and colloidal silicon dioxide.

More preferably, the lubricant is selected from magnesium stearate, zinc stearate, calcium stearate and colloidal silicon dioxide.

Prefearbly, the oral pharmaceutical composition comprises amorphous darunavir, prosolv, crospovidone, colloidal silicon dioxide and magnesium stearate.

The wet granulation process includes wet granulation of amorphous darunavir with the excipient(s), lubrication and followed by compression.

The compaction process includes compaction of amorphous darunavir with the excipient(s), lubrication and followed by compression.

The direct compression process includes blending amorphous darunavir with the excipient(s), lubrication and followed by compression.

The tablet composition is optionally film coated with opadry II orange.

The following examples further exemplify the invention and are not intended to limit the scope of the invention.

### Example 1

| Ingredients | Quantity/Unit (mg) |
|---|---|
| Amorphous darunavir* | 600.00 |
| Prosolv | 583.75 |
| Crospovidone | 37.50 |
| Purified water | q.s |
| Colloidal silicon dioxide | 25.00 |
| Magnesium stearate | 3.75 |
| Film coating | |
| Opadry II orange | 25.00 |
| Total Tablet weight | 1275.00 |

*Particle size distribution of amorphous darunavir: d₁₀-11 μm; d₅₀-50 μm d₉₀-195 μm

The process of the preparation involve following steps:

i). Blending of amorphous darunavir, prosolv, crospovidone dried if necessary

ii). Lubricating with colloidal silicon dioxide and magnesium stearate

iii). Compressing the lubricated blend of step (ii) into tablets.

iv). Coating the compressed tablets with opadry.

### Example 2

| Ingredients | Quantity/Unit (mg) |
|---|---|
| Amorphous darunavir* | 600.00 |
| Prosolv | 596.25 |
| Crospovidone | 25.00 |
| Purified water | q.s |
| Colloidal silicon dioxide | 25.00 |
| Magnesium stearate | 3.75 |
| Film coating | |
| Opadry II orange | 25.00 |
| Total Tablet weight | 1275.00 |

*Particle size distribution of amorphous darunavir: d₁₀-15 μm; d₆₀-60 μm d₉₀-210 μm

The process of the preparation involve following steps:

i). Granulation of amorphous darunavir, prosolv, crospovidone with a suitable sovent.

ii). Lubricating with colloidal silicon dioxide and magnesium stearate

iii). Compressing the lubricated blend of step (ii) into tablets.

iv). Coating the compressed tablets with opadry.

### Example 3

| Ingredients | Quantity/Unit (mg) |
|---|---|
| Amorphous darunavir* | 600.00 |
| Prosolv | 608.75 |
| Crospovidone | 25.00 |
| Purified water | q.s |
| Colloidal silicon dioxide | 12.50 |
| Magnesium stearate | 3.75 |
| Film coating | |
| Opadry II orange | 25.00 |
| Total Tablet weight | 1275.00 |

*Particle size distribution of amorphous darunavir: d₁₀-17 μm; d₅₀-65 μm d₉₀-213 μm

The process of the preparation involve following steps:

i). Compaction of Darunavir, prosolv, crospovidone with a suitable sovent.

ii). Lubricating with colloidal silicon dioxide and magnesium stearate

iii). Compressing the lubricated blend of step (ii) into tablets.

iv). Coating the compressed tablets with opadry.

### Example 4

| Ingredients | Quantity/Unit (mg) |
|---|---|
| Amorphous darunavir* | 600.00 |
| Microcrystalline cellulose | 571.75 |
| Crospovidone | 37.50 |
| Purified water | q.s |
| Colloidal silicon dioxide | 37.00 |
| Magnesium stearate | 3.75 |
| Film coating | |
| Opadry II orange | 25.00 |
| Total Tablet weight | 1275.00 |

*Particle size distribution of amorphous darunavir: d₁₀-8 μm; d₅₀-45 μm d₉₀-182 μm

The process of the preparation involve following steps:

i). Blending of amorphous darunavir, microcrystalline cellulose, crospovidone and dried if necessary

ii). Lubricating with colloidal silicon dioxide and magnesium stearate

iii). Compressing the lubricated blend of step (ii) into tablets.

iv). Coating the compressed tablets with opadry.

We claim:

1. An oral pharmaceutical composition comprises amorphous darunavir having a d₉₀ particle size in the range of about 150 μm to 250 μm.

2. The oral pharmaceutical composition according to claim 1, wherein the d₉₀ particle size is in the range of 175 to 225 μm.

3. The oral pharmaceutical composition according to claim 1, wherein d₉₀ particle size is in the range of 190 to 200 μm.

4. The oral pharmaceutical composition according to claim 1, wherein the composition is in the form of tablets.

5. The oral pharmaceutical composition according to claim 4, wherein the tablet is film coated.

6. The oral pharmaceutical composition according to claim 1, wherein the composition may contain one or more additional excipients.

7. The oral pharmaceutical composition according to claim 6, the excipients are selected from diluents, binders, disintegrants and lubricants.

8. The oral pharmaceutical composition according to claim 7, wherein the diluent is selected from lactose, sucrose, glucose, mannitol, sorbitol, calcium carbonate, microcrystalline cellulose, Prosolv, magnesium stearate and mixtures thereof.

9. The oral pharmaceutical composition according to claim 7, wherein the diluent is Prosolv.

10. The oral pharmaceutical composition according to claim 7, wherein the binder is selected from L-Hydroxy propyl cellulose, polyvinyl pyrrolidine, hydroxyl propyl methyl cellulose, hydroxyl ethyl cellulose and pre-gelatinized starch.

11. The oral pharmaceutical composition according to claim 7, wherein the disintegrant is selected from croscarmellose sodium, crospovidone, sodium starch glycolate and low substituted hydroxyl propyl cellulose.

12. The oral pharmaceutical composition according to claim 7, wherein the disintegrant is selected from low substituted hydroxyl propyl cellulose and crospovidone.

13. The oral pharmaceutical composition according to claim 7, wherein the lubricant is selected from sodium stearyl fumarate, magnesium stearate, zinc stearate, calcium stearate, stearic acid, talc, Glyceryl behenate and colloidal silicon dioxide.

14. The oral pharmaceutical composition according to claim 7, wherein the lubricant is selected from magnesium stearate, zinc stearate, calcium stearate and colloidal silicon dioxide.

15. The oral pharmaceutical composition according to claim 1, wherein the tablets of darunavir comprises amorphous darunavir, colloidal silicon dioxide, crospovidone, magnesium stearate and prosolv.
